(19) United States
(12) Patent Application Publication
(10) Pub. No.: US 2024/0020842 A1
(43) Pub. Date: Jan. 18, 2024

(54) SYSTEMS AND METHODS FOR IMAGE ALIGNMENT AND REGISTRATION

(71) Applicant: Washington University, St. Louis, MO (US)

(72) Inventors: Graham Colditz, St. Louis, MO (US); Shu Jiang, St. Louis, MO (US)

(73) Assignee: Washington University, St. Louis, MO (US)

(21) Appl. No.: 18/353,913

(22) Filed: Jul. 18, 2023

Related U.S. Application Data
(60) Provisional application No. 63/390,212, filed on Jul. 18, 2022.

(51) Int. Cl. G06T 7/00; G06T 7/33; G06T 7/11; G06T 7/12; G16H 30/40; G16H 50/30; G06T 3/00

(52) U.S. Cl. CPC G06T 7/0014; G06T 7/337; G06T 7/11; G06T 7/12; G16H 30/40; G16H 50/30; G06T 3/0068; G06T 2207/20021; G06V 10/26

(57) ABSTRACT

Among the various aspects of the present disclosure are the provision of an image alignment and registration system and a breast cancer risk prediction system.

### CROSS-REFERENCE TO RELATED APPLICATIONS

This application claims priority from U.S. Provisional Application Ser. No. 63/390,212 filed on Jul. 18, 2022, the content of which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to an image alignment and registration system.

### BACKGROUND OF THE INVENTION

With the exponential growth in image data collection, more advanced analyses are focusing on making full use of mammogram images to improve personalized breast cancer risk prediction. The variation in processed images, such as breast size and position, are present even within the same set of images taken over time for the same individual. This variation makes the identification and monitoring of regions of interest over time (such as tracking tumor evolution over 5 years) burdensome, as it involves hand-matching and eyeballing a series of mammograms that invariably introduces inconsistencies among clinicians. To ensure high-quality results from various image analysis methods, multiple images must be aligned/registered on the same coordinate system prior to any analytical procedures to avoid estimation bias and variation. However, no well-accepted tool in the field for mammogram registration and alignment exists at present.

Breast cancer is the leading cancer diagnosed among women worldwide accounting for more than 1 in 4 cancers diagnosed and is increasing globally. Risk stratification to tailor prevention strategies for this common malignancy is urgently needed to guide prevention and early detection to combat this disease burden.

The use of mammography for early detection of breast cancer is widespread and both age at initiation and screening interval vary across countries. In the USA, mammography data from 2018 show that 72 to 75% of women aged 50 to 74 have had a mammogram in the past 2 years.

The leading measure for long-term risk categorization extracted from mammograms is breast density, shown illustrated in FIG. 1. Mammographic breast density (MD) is a strong reproducible risk factor for breast cancer across different measurement approaches, such as clinical judgment or semi-automated estimation, and across patient populations in different regions of the world. Breast density decreases starting at about age 30 and this decrease is strongly influenced by menopause. The consistency of this decrease across countries and races leads to the conclusion that breast density is a universal biologic mechanism serving as an intermediate marker of breast cancer risk. Texture features within mammograms add richness to details beyond MD but have been much less frequently studied for their contribution to risk stratification and risk prediction.

In current medical practice, risk prediction analysis methods provide objective ways to assess a patient's risk of developing a disease, such as a 10-year risk of cardiovascular disease. Historically, breast cancer prediction models either made use of reproductive and other questionnaire-based risk factors, or focused on identifying high-risk genetic markers. The predictive ability of questionnaire-based risk factors was enhanced by adding mammographic breast density and polygenic risk scores. Despite merging data from these more complex data sources, the prediction AUC typically does not exceed 0.72. Numerous studies report an association with breast cancer for various texture features extracted by hand, by automation, and by machine learning methods. These approaches are not consistent across studies and, like MD, make use of only a relatively small fraction of the information contained within the mammogram image, leaving approximately 13 million pixels per image largely unused.

Recently, deep learning (DL) approaches have been developed to facilitate the diagnosis of breast cancer and have been extended to implement risk prediction in some cases. When comparable populations are used that exclude cases diagnosed in the first 6 months after entry, the 5-year prediction performance (AUC) in these DL models ranges from 0.70 to 0.72.

### SUMMARY OF THE INVENTION

Among the various aspects of the present disclosure are the provision of an image alignment and registration system and a breast cancer risk prediction system.

In one aspect, a system for aligning and registering a medical image with a reference medical image is disclosed that includes at least one processor in communication with at least one memory device. The at least one processor is programmed to receive the medical image and a reference image; convert the medical image to a binary image; isolate an area of interest within the medical image to produce an isolated image; remove at least one portion of the isolated image containing at least one user-selected tissue type to produce a segmented image; flip or rotate the segmented image into alignment with the reference image to produce an aligned image; and register the aligned image to the reference image to produce an aligned and registered image. In some aspects, the medical image is selected from a longitudinal series of medical images and the reference image comprises an initial medical image of the series. In some aspects, the medical image is selected from a dataset comprising a plurality of medical images obtained from a plurality of subjects and the reference image comprises a user-selected medical image from the dataset. In some aspects, the medical image is selected from a digital mammogram image and at least a portion of a digital 3D tomosynthesis image. In some aspects, the medical image further comprises a craniocaudal view or a mediolateral oblique view. In some aspects, the area of interest of the medical image comprises a portion of the medical image containing a breast region. In some aspects, the area of interest is isolated by fitting a rectangle of minimal dimension around the breast region. In some aspects, the at least one user-selected tissue type removed from the isolated image comprises soft tissues outside of the breast region within craniocaudal views, pectoral muscle tissue within mediolateral oblique views, and any combination thereof. In some aspects, the at least one processor is further programmed to automatically determine the soft tissues outside the breast region based on a union of discontinuities on a boundary of the breast area and deviations from a semi-circular shape, wherein the semicircular shape is selected to approximate the boundary of the breast area. In some aspects, the at least one processor is further programmed to automatically determine the pectoral muscle tissue by binarizing the medical image, applying a Canny algorithm to detect an outer edge of the breast tissue, and removing a portion of the image falling outside of the outer edge of the breast tissue. In some aspects, the at least one processor is further programmed to produce the aligned image by finding a width ratio between the segmented image and the reference image; obtaining an alignment angle between a line along the top of the segmented image and a line connecting the top left corner and the largest horizontal (x) point of the breast tissue within the segmented image; rotating the segmented image to align the alignment angle with a corresponding alignment angle of the reference image. In some aspects, the at least one processor is further programmed to register the aligned image to the reference image by adjusting a ratio in image width pixelwise between the aligned image and the reference image. In some aspects, the at least one processor is further programmed to: identify an abnormal region within one medical image from the longitudinal series of medical images; identify a monitor region for each medical image of the longitudinal series of medical images, wherein the monitor region of each medical image is matched to the abnormal region of the one medical image; and display a series of monitor images to a user, the series of monitor images comprising the longitudinal series of medical images demarcated with each corresponding abnormal region or monitor region. In some aspects, the at least one processor is further programmed to display magnified views of the abnormal region and monitor regions to the user. The at least one processor is further programmed to: identify text within the medical image; and determine a view of the binary image based on the identified text, wherein the view is a craniocaudal view or a mediolateral oblique view.

In another aspect, a system for predicting the risk of breast cancer of a patient from analysis of a medical image is disclosed. The system includes at least one processor, the at least one processor configured to: transform the medical image into a characterized image by forming bivariate splines over a two-dimensional triangulated domain of the medical image; perform a survival analysis of the characterized image to obtain a prediction of the risk of breast cancer in the patient; and display the prediction of the risk of breast cancer to a practitioner. In some aspects, the at least one processor is further configured to form bivariate splines over a two-dimensional triangulated domain of the medical image by forming the two-dimensional triangulated domain using Delaunay Triangulation and forming the bivariate splines using a Bernstein polynomial basis function. In some aspects, the at least one processor is further configured to perform a survival analysis of the characterized imaging using a model selected from a right-centered survival model and a Cox proportional hazards model. In some aspects, the medical image is a mammogram.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 contains randomly selected mammograms categorized as BI-RADS categories A, B, C, and D. The purple bar indicates the percentage of women in the Joanne Knight Breast Health Cohort composed of 10,092 women that are in the corresponding BI-RADS 4th edition category. The red bar shows the category-specific percentage of breast cancer incidence.

FIG. 2A is a schematic overview of a portion of FLIP including the initial formation of the characterized image with bivariate splines over triangulation that is processed further as described in FIG. 2B and FIG. 2C. The raw images are in the form of .dcm files before entering into FLIP. After automated processing and image alignment, the two CC-views (left and right) are averaged between the two breasts for characterization. The inputted 2D mammograms are first characterized with bivariate splines over triangulation to preserve the spatial distribution of pixels and accommodate the irregular semi-circular breast boundary. The characterization is further optimized as described above, which provides a unique and closed-form solution.

FIG. 2B is a schematic overview of a portion of FLIP including the inclusion of the characterized image described in FIG. 2A within a Cox proportional hazards model. A simple Cox proportional hazards model is adopted using well-established risk factors (RF), including age, breast density (BI-RADS), BMI, menopausal status, parity, family history, and history of benign breast disease. The mammogram image acts as an additional risk factor in the Cox regression accompanied with a 2D coefficient surface. All inferential procedures with Cox regression are applicable to FLIP which provides a transparent workflow ensuring high reproducibility. hᵢ(t) denotes the hazard function at time t for individual i, and h₀(t) denotes the nonparametric baseline hazard function.

FIG. 2C is a representative graph of a survival curve that is generated using the Cox regression model described in FIG. 2B. Women who were diagnosed with breast cancer within the first 6 months of their mammogram date have been removed from this analysis and the model focused on the 5-year risk. Discriminatory performance was assessed with AUC and validated via a 10-fold cross-validation.

FIG. 3A is a triangulation grid for mammograms using 87 triangles.

FIG. 3B is a triangulation grid for mammograms using 115 triangles.

FIG. 3C is a triangulation grid for mammograms using 147 triangles.

FIG. 4 is an Illustration of a Bernstein polynomial basis function with r=1, d=2.

FIG. 5 is a block diagram schematically illustrating a system in accordance with one aspect of the disclosure.

FIG. 6 is a block diagram schematically illustrating a computing device in accordance with one aspect of the disclosure.

FIG. 7 is a block diagram schematically illustrating a remote or user computing device in accordance with one aspect of the disclosure.

FIG. 8 is a block diagram schematically illustrating a server system in accordance with one aspect of the disclosure.

FIG. 9A is a predicted survival curve for two women randomly selected from the testing set with BI-RADS category D. Individual 1 (red): age=56.54, BMI=27.46, postmenopausal, parous=1, history of benign breast disease (BBD)=1, family history (fh)=0; Individual 2 (purple): age=59.1, BMI=25.33, postmenopausal, parous=0, BBD=1, fh=1; Both individuals are white.

FIG. 9B shows the left and right mammograms corresponding to the two individuals in FIG. 9A with BI-RADS category D at the baseline.

FIG. 9C is a predicted survival curve for two individuals in the testing set with BI-RADS category B. Individual 1 (red): age=68.23, BMI=31.24, postmenopausal, parous=1, BBD=0, fh=0; Individual 2 (purple): age=49.09, BMI=33.28, postmenopausal, parous=1, BBD=0, fh=0; Individual 1 (red) is white and individual 2 (purple) is black.

FIG. 9D shows the left and right mammograms that correspond to the two individuals in FIG. 9C with BI-RADS category B at the baseline.

FIG. 10A is a digital mammogram as originally recorded.

FIG. 10B is the mammogram of FIG. 10A with the automatically detected text label highlighted as a colored area on the right side of the panel.

FIG. 11 is the mammogram of FIG. 10A after automatically enclosing the breast region using a tight rectangular box.

FIG. 12 contains unmodified serial mammograms for both the LCC (top row) and RCC (bottom row) views before alignment (raw images).

FIG. 13 contains the serial mammogram images of FIG. 12 after alignment and registration using the systems and methods disclosed hererin; green represents the reference/original image and purple represents the moving/subsequent image.

FIG. 14 is a schematic illustration showing a method of tracing back regions of interest in a series of longitudinal mammogram images using the systems and methods disclosed herein.

FIG. 15 is a block diagram illustrating a method of aligning and registering a mammogram image in accordance with one aspect of the disclosure.

FIG. 16A is an example of a mammogram image before an application of the Canny algorithm for edge detection.

FIG. 16B is an example of a mammogram image after the application of the Canny algorithm for edge detection.

FIG. 17 is an image showing the algorithm-detected edge for the breast region.

FIG. 18A is an image of an original mammogram.

FIG. 18B is an image of the mammogram in FIG. 18A wherein the green line represents the true pectoral muscle region on the mammogram. The red line illustrates the false positive regions (FP) and false negative regions (FN).

FIG. 19 is an example of pectoral muscle identification in a mammogram. The first column represents the true pectoral muscle region as compared to regions identified using the disclosed algorithm (second column) and using the Libra algorithm (third column).

FIG. 20 is another example of pectoral muscle identification in a mammogram. The first column represents the true pectoral muscle region as compared to regions identified using the disclosed algorithm (second column) and using the Libra algorithm (third column).

FIG. 21 is a table of the estimated false positive (FP) and false negative classifications for both the left and right MLO.

FIG. 22 is an image showing a representative alignment line and angle superimposed over a mammogram.

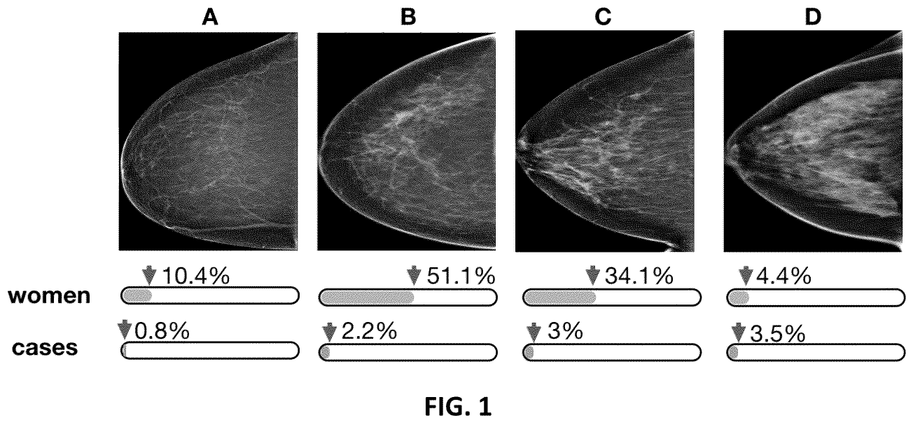

### DETAILED DESCRIPTION OF THE INVENTION

One aspect of the system and method is a feature that allows a user to save a high-quality registered image that is approximately 7 times smaller than the original mammogram .dicom images. In some embodiments, the disclosed data alignment and registration method may result in a significant reduction in the resources dedicated to the storage of mammogram images. In some aspects, the registered images produced using the disclosed systems and methods may be capable of storage on a patient's storage media for use by any practitioner of the patient's choosing without the need for image access via institutionally curated large-scale medical image storage systems.

In various aspects, automated systems and methods for aligning and registering serial digital 2D mammograms and 3D digital breast tomosynthesis images on a reference coordinate system are disclosed hererin. In some aspects, the disclosed systems and methods provide for accurate and efficient tracking of regions of interest from personalized longitudinal mammogram images in the clinical setting. The aligned images can be used as a means of diagnosis, prognosis, identification of tumors, characterization of breast tissue, risk stratification, and long-term risk prediction.

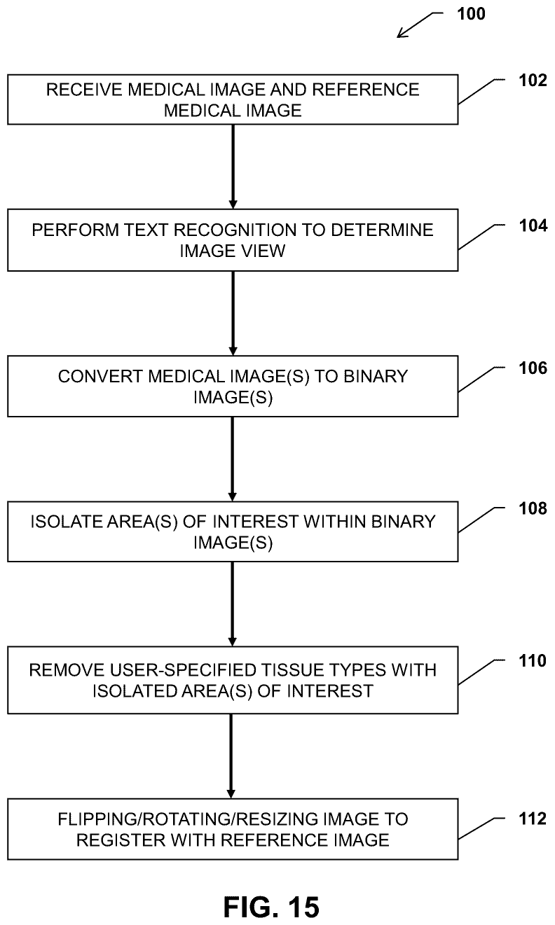

FIG. 15 is a block diagram illustrating the steps of an automated method 100 for aligning and registering medical images including, but not limited to, serial digital 2D mammograms in various aspects. The method 100 comprises receiving a medical image and a reference medical image including, but not limited to, mammograms at 102. The medical image and reference medical image may be provided in any suitable format known in the art without limitation including mammograms provided in a .dicom format. In some aspects, the reference mammogram and the mammogram comprise an initial mammogram and a subsequent mammogram of a longitudinal series obtained from a single subject over time, respectively. In other aspects, the reference mammogram comprises a selected mammogram from an image dataset including, but not limited to, an image registry, and the mammogram comprises a mammogram of one patient from a population of patients from the image dataset or other collection of mammograms. In other additional aspects, the reference mammogram comprises a mammogram of a healthy or control subject and the mammogram comprises a mammogram obtained from a subject diagnosed or suspected to have a breast tissue anomaly.

In various aspects, any suitable medical image of breast tissue may be received at 102 including, but not limited to, mammograms, planar sections of 3D digital breast tomosynthesis images, planar slices of MRI images, X-ray images, planar slices of CT images, and images obtained using any other suitable medical imaging modality. In some aspects, the planar sections of the 3D digital breast tomosynthesis images and other 3D imaging modalities may be matched between the reference image and the image to be aligned and registered such that both images are within a coincident plane. In various other aspects, the view or orientation of the reference mammograms and mammograms are matched. Any suitable mammogram view or orientation may be used in the disclosed method without limitation including, but not limited to, craniocaudal, and mediolateral oblique.

It is noted that although the disclosed systems and methods are generally described herein in terms of mammograms, the disclosed systems and methods may be modified and used to align and analyze a variety of other breast images obtained using a variety of imaging modalities. Non-limiting examples of breast images that may be aligned and analyzed using the systems and methods disclosed herein include full-field digital mammography, digital breast tomosynthesis (DBT) synthetic digital mammography generated from DBT, MRI, and CT scans.

It is further noted that although the disclosed systems and methods are generally described herein in terms of breast images, the disclosed systems and methods are compatible, with minimal modification, align and analyze images of a variety of other organs including liver images and lung images.

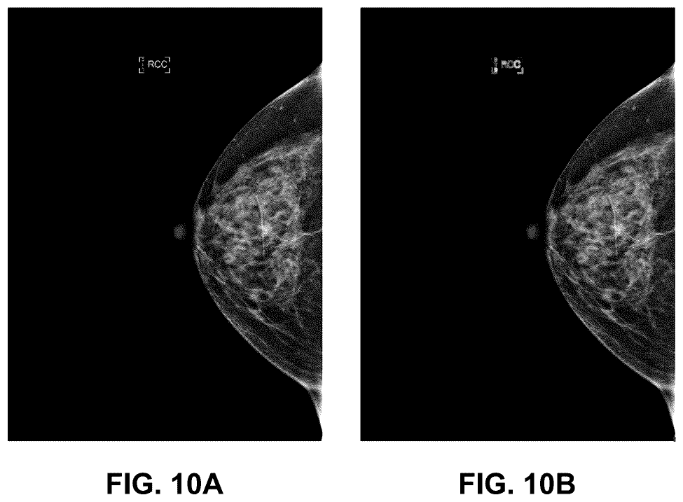

Referring again to FIG. 15, the method 100 further includes performing text recognition to determine the view of the reference mammogram and mammogram at 104. In various aspects, the text label included on the mammogram (see FIG. 10A) is indicative of the orientation of the image as well as whether the image was obtained from a left or right breast. Any suitable automated method may be used to perform the text recognition at 104 without limitation. In some aspects, maximally stable extremal regions (MSER), a technique used in computer vision, is used for blob detection in the mammograms. Given the MSER region, an aspect ratio using bounding box data is estimated. Thresholding and stroke width variation are also performed to remove regions within the MSER region that do not contain text information. With the cropped text area, connected graph components are identified to recognize the text on the mammogram. A binary output specifying whether the connected text regions are contained within the pre-specified vision text is returned. For example, if the string “RCC” indicating a right-side craniocaudal view (see FIG. is detected in the mammogram, both the view and the type of mammogram are specified. In some aspects, subsequent transformations of the mammogram images, including, but not limited to, image rotation/flipping and/or soft tissue removal as described below are selected in the subsequent pipeline based on the type/view of mammogram identified using the automated text recognition at 104. In some aspects, the recognized text is removed from the medical image prior to further analysis.

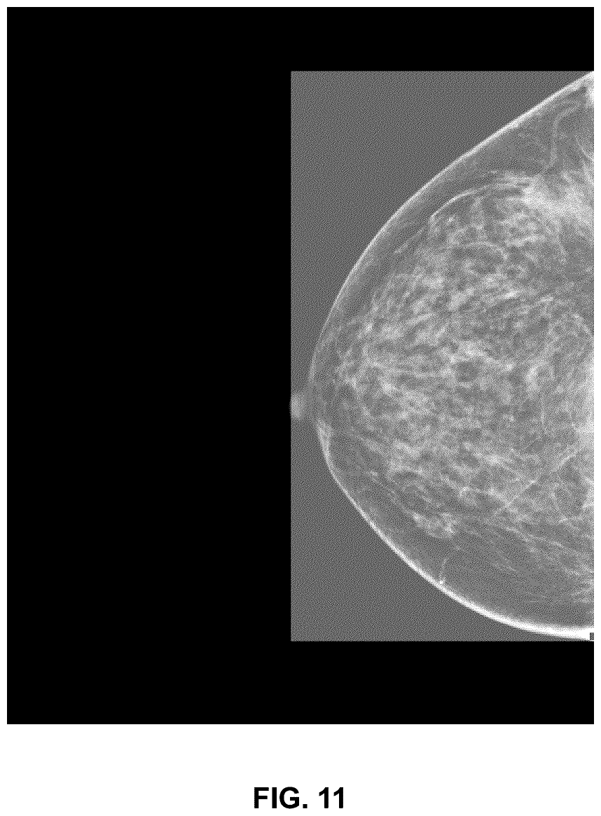

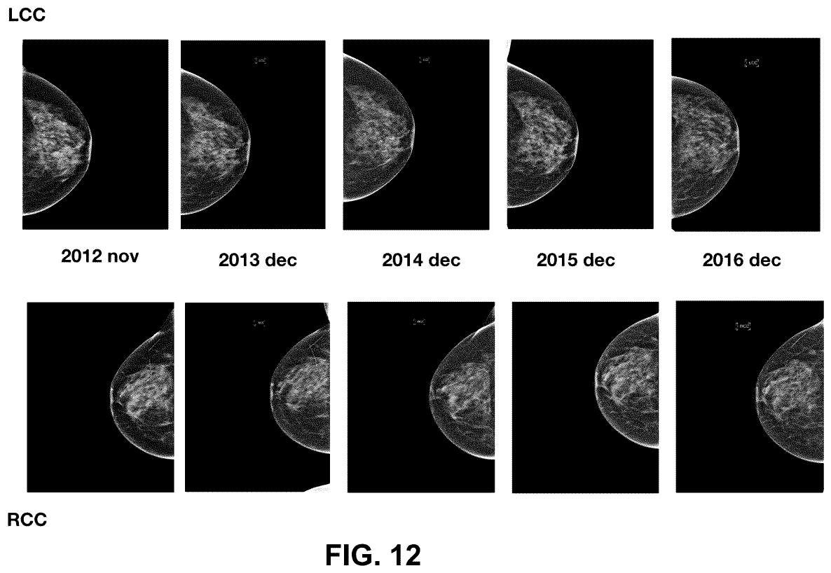

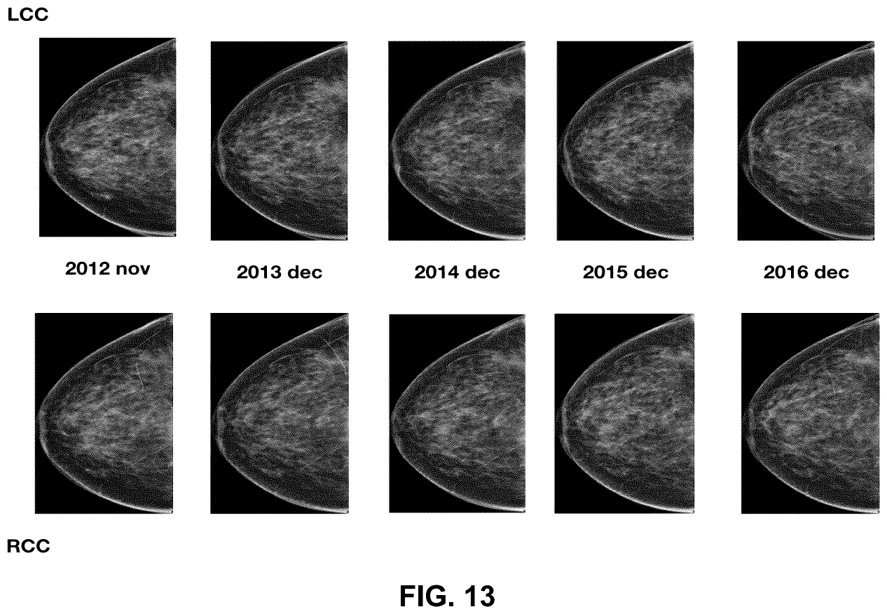

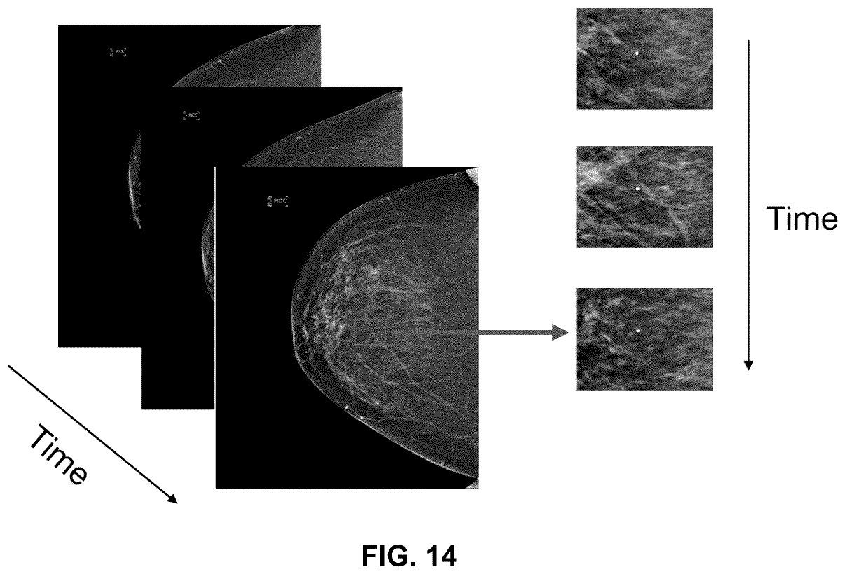

Referring again to FIG. 15, the method 100 may further include converting the medical images to binary images at 106 and identifying the areas of interest within the medical images at 108. In various aspects, the medical images are automatically converted to binary images at 106 using any suitable method known in the art without limitation. In various other aspects, the area of interest is automatically identified using any suitable method known in the art without limitation including, but not limited to, determining the smallest box that includes the breast region as illustrated in FIG. 11.

Referring again to FIG. 15, the method 100 may further include removing at least one portion of the isolated image containing at least one user-selected tissue type to produce a segmented image at 110. Any tissue may be selected by a user for removal without limitation including, but not limited to, a soft tissue such as muscle tissue. In other aspects, tissues may be selected for removal based on the type and view of the medical image as determined by text recognition at 104 in some aspects. For craniocaudal views, soft tissues outside of the breast regions are automatically determined by the union of discontinuities on the boundary of the breast area and deviations from the semi-circular shape in some aspects.

In some aspects, pectoral muscles are removed from mediolateral oblique views by determining the linear plane on the image separated by a blob of continuous high pixel intensities that are clustered together in some aspects. In other aspects, the pectoral muscles are removed from mediolateral oblique views by binarizing the image as described above, applying a Canny algorithm to detect the outer edge of the breast tissue, and removing the portion of the image falling outside of the breast tissue edge. A description of the Canny algorithm may be found in Ding L, Goshtasby A: “On the Canny edge detector.” Pattern recognition 2001, 34(3):721-725, the content of which is incorporated by reference in its entirety. In some additional aspects, the breast tissue edge identified by the Canny algorithm, which may be in a rough and pixelated form, may be smoothed using a robust smoothing algorithm. A non-limiting example of a suitable robust smoothing algorithm may be found at Fischler M A, Bolles R C: “Random sample consensus: a paradigm for model fitting with applications to image analysis and automated cartography.” Communications of the ACM 1981, 225 24(6):381-395, the content of which is incorporated by reference in its entirety.

Referring again to FIG. 15, the method 100 may further include flipping or rotating the segmented image into alignment with a reference image to produce an aligned image and registering the aligned image to a user-selected image size to produce an aligned and registered image at 112. In some aspects, alignment is performed using a bicubic interpolation based on a weighted average of pixels in a nearest 4-by-4 neighborhood to a user-selected image size of X×Y.

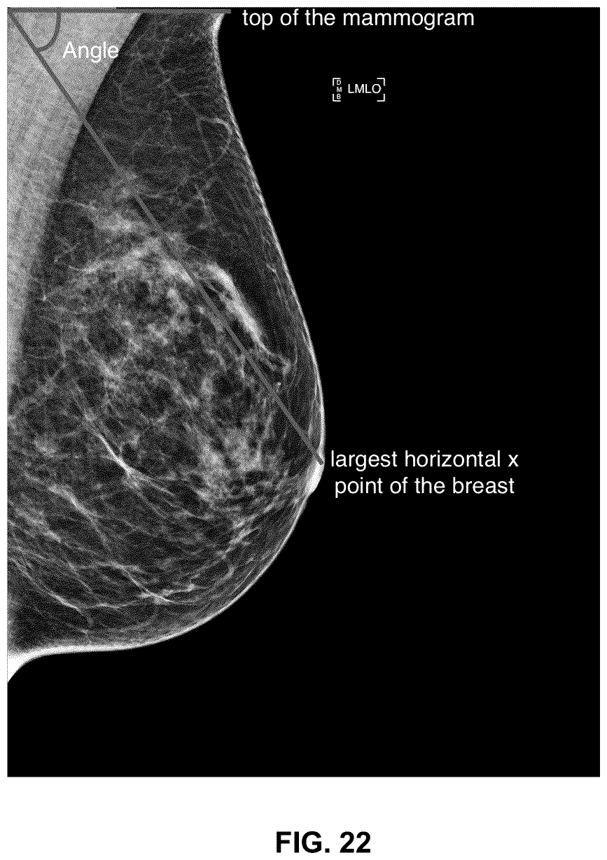

In other aspects, the medical image is aligned with the segmented image by finding a width ratio between the two images, and then defining an alignment angle between a line along the top of the mammogram and a line connecting the top left corner of the mammogram and the largest horizontal (x) point of the breast tissue within the mammogram image. FIG. 22 shows a representative alignment line and angle as described above, The segmented image may then be rotated so that the line defined in the segmented image aligns with the corresponding line defined in the reference image.

In various aspects, after the alignment of the segmented and reference images as described above, the registration of the segmented image with the reference image is performed pixel by pixel by adjusting the ratio in image width of the two images without altering or interpolating any values on the images. In various aspects, the user-selected image size may be any suitable size without limitation. In some aspects, the user-selected image size comprises X x Y, wherein X ranges from about 1 pixel to about 5000 pixels and Y ranges from about 1 pixel to about 5000 pixels. In various other aspects, X and Y are independently selected to be at least 1 pixel, at least 10 pixels, at least 20 pixels, at least 30 pixels, at least 40 pixels, at least 50 pixels, at least 100 pixels, at least 200 pixels, at least 300 pixels, at least 400 pixels, at least 500 pixels, at least 1000 pixels, at least 2000 pixels, at least 3000 pixels, at least 4000 pixels, and at least 5000 pixels. In various additional aspects, X and Y are independently selected no more than 10 pixels, no more than 20 pixels, no more than 30 pixels, no more than 40 pixels, no more than 50 pixels, no more than 100 pixels, no more than 200 pixels, no more than 300 pixels, no more than 400 pixels, no more than 500 pixels, no more than 1000 pixels, no more than 2000 pixels, no more than 3000 pixels, no more than 4000 pixels, and no more than 5000 pixels, wherein X ranges from about 100 pixels to about 1000 pixels and Y ranges from about 100 pixels to about 2000 pixels. In one aspect, the user-selected image size is 500 pixels×800 pixels.

In various other aspects, the method may further include various additional steps to analyze and/or display the registered images to facilitate the diagnosis of a disorder, select a treatment, monitor the progression of a disorder, monitor the efficacy of a treatment, or any other suitable form of analysis or display of one or more registered images. In some aspects, the registered image may be analyzed to identify an abnormal region within one medical image from the longitudinal series of medical images. In other aspects, a monitor region may be identified for each medical image of the longitudinal series of medical images, wherein the monitor region of each medical image is matched to the abnormal region of the one medical image. In other additional aspects, the system may display a series of monitor images to a user, wherein the series of monitor images include the longitudinal series of medical images demarcated with each corresponding abnormal region or monitor region. In some aspects, the system may display magnified views of abnormal regions and/or monitor regions to the user.

In some embodiments, the modeling framework can be utilized in designing prevention clinical trials for sample size and power derivations. In some embodiments, the modeling framework's transparent workflow for image characterization enables inferential procedures including but not limited to evaluating associations of predictors to the whole image, including questionnaire-based breast cancer risk factors, SNPs, and novel or emerging biomarkers. In some embodiments, the extent to which the effect of risk factors is mediated through the mammogram images and the extent it is through other pathways is determined.

In some embodiments, multiple images are taken over time and analyzed. In some embodiments, repeated mammographic images are analyzed to stratify risk or identify high-risk groups or low-risk groups to tailor screening and prevention. In some embodiments, the risk is determined by changes in risk factors over time and changes in analyzed images over time. In some embodiments, the images are whole mammograms. In some embodiments, patients can be cancer patients. In some embodiments, patients can be breast cancer patients. In some embodiments, patients can be invasive breast cancer patients. In some embodiments, the system identifies patients for more intensive prevention. In some embodiments, the system decreases the burden on women in terms of collecting additional risk factors and biologic samples to generate polygenic risk scores and related parameters compared to current models. In some embodiments, the system removes the barriers to wider clinical use without prohibitive training data and extensive computational requirements. In some embodiments, the system provides a transparent workflow ensuring high reproducibility. In some embodiments, the workflow can be performed on a standard desktop without parallel computing.

In some embodiments, the system and methods provide 5- and 10-year risk stratification in cancer patients. In some embodiments, the patients are breast cancer patients. In some embodiments, the risk stratification can be applied in real-time in the clinical setting maximizing benefit-to-harm ratio. In some embodiments, the risk assessment can occur in less than 7 minutes.

In some embodiments, the 5-year prediction performance of the system exceeds models drawing data from multiple sources (questionnaires data, SNPs, and MD). In some embodiments, the 5-year prediction performance exceeds that of models using similar eligibility criteria and follow-up and models that include a broader range of epidemiologic risk factors. In some embodiments, the patient data is from breast cancer patients. In some embodiments, the 5-year prediction model is refined with the inclusion of risk factors, including but not limited to the history of benign breast biopsy, weight change, use of combination estrogen plus progestin, race, and menopausal status. In some embodiments, routine clinical genomics and metabolomics can be integrated into the system. In some embodiments, data from multiple sources, including but not limited to questionnaires or electronic medical records, saliva or blood for DNA, and mammograms, are integrated into the system to generate personalized risk classification. In some embodiments, the 5-year prediction model incorporates changes in risk factors.

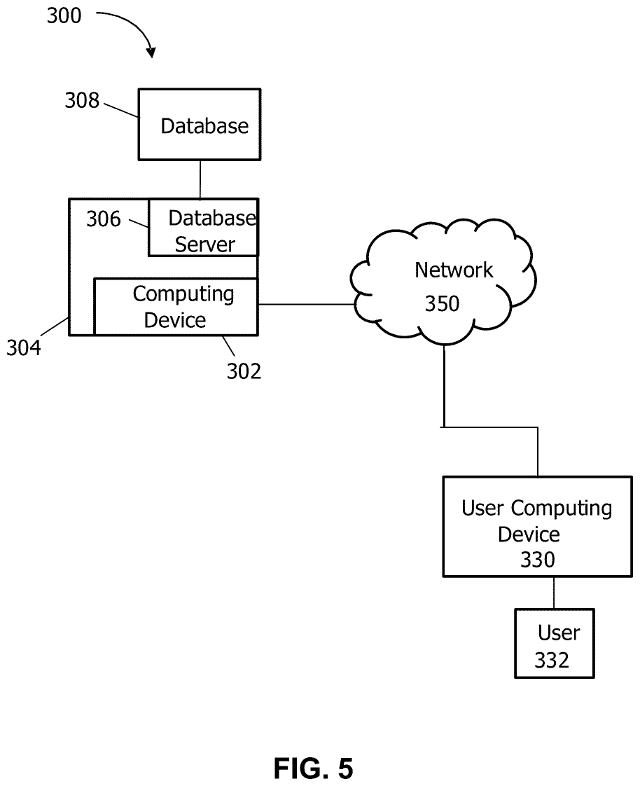

In various aspects, at least a portion of the methods disclosed herein may be implemented using various computing systems and devices as described below. FIG. 5 depicts a simplified block diagram of a computing device for implementing the image analysis methods described herein. As illustrated in FIG. 5, the computing device 300 may be configured to implement at least a portion of the tasks associated with the systems and methods for aligning and registering medical images. The computer system 300 may include a computing device 302. In one aspect, the computing device 302 is part of a server system 304, which also includes a database server 306. The computing device 302 is in communication with a database 308 through the database server 306. The computing device 302 is communicably coupled to a user-computing device 330 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user-computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

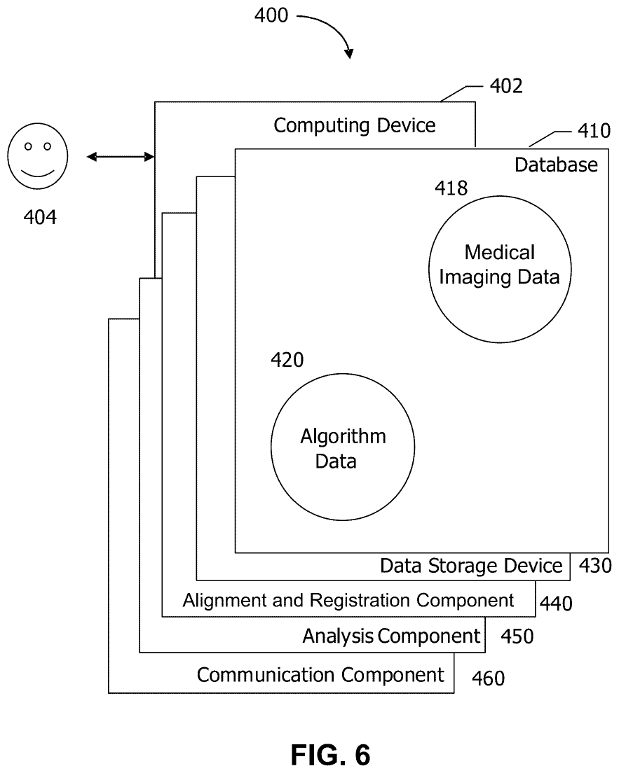

In other aspects, the computing device 302 is configured to perform a plurality of tasks associated with the medical image alignment and registration methods described herein. FIG. 6 depicts a component configuration 400 of computing device 402, which includes database 410 along with other related computing components. In some aspects, computing device 402 is similar to computing device 302 (shown in FIG. 5). A user 404 may access components of computing device 402. In some aspects, database 410 is similar to database 308 (shown in FIG. 5).

In one aspect, database 410 includes medical imaging data 418 and algorithm data 420. Non-limiting examples of mammogram data 418 include any data associated with medical images or subsequently processed data including, but not limited to, the medical images, corresponding binary images, and aligned and registered images. non-limiting examples of medical images include mammograms, planar sections of 3D digital breast tomosynthesis images, planar slices of MRI images, X-ray images, planar slices of CT images, and images obtained using any other suitable medical imaging modality. Non-limiting examples of suitable algorithm data 420 include any values of parameters defining the alignment and registration of the medical images according to the methods disclosed herein. Other non-limiting examples of suitable algorithm data 420 include any parameters defining the user-selected image size, the boundary of the breast area, the rectangle of minimal dimension, the view of the medical image, and any other parameter relevant to the methods of alignment and registration of medical images described herein.

Computing device 402 also includes a number of components that perform specific tasks. In the exemplary aspect, computing device 402 includes a data storage device 430, an alignment and registration component 440, an analysis component 450, and a communication component 460. The data storage device 430 is configured to store data received or generated by computing device 402, such as any of the data stored in database 410 or any outputs of processes implemented by any component of computing device 402. The alignment and registration component 440 is configured to align and register medical images using the methods disclosed herein.

The analysis component 450 is configured to analyze the aligned and registered medical images as disclosed herein. In some aspects, the analysis component 450 may identify an abnormal area within one medical image from a series of longitudinal medical images and trace the corresponding regions in one or more adjoining medical images in the series of longitudinal medical images for display to a user. In other aspects, the analysis component 450 may stratify risk or identify high-risk groups or low-risk groups to tailor screening and prevention based on comparisons of aligned and registered medical images using methods described herein.

Communication component 460 is configured to enable communications between computing device 402 and other devices (e.g. user computing device 330 shown in FIG. 5) over a network, such as network 350 (shown in FIG. 5), or a plurality of network connections using predefined network protocols such as TCP/IP (Transmission Control Protocol/Internet Protocol).

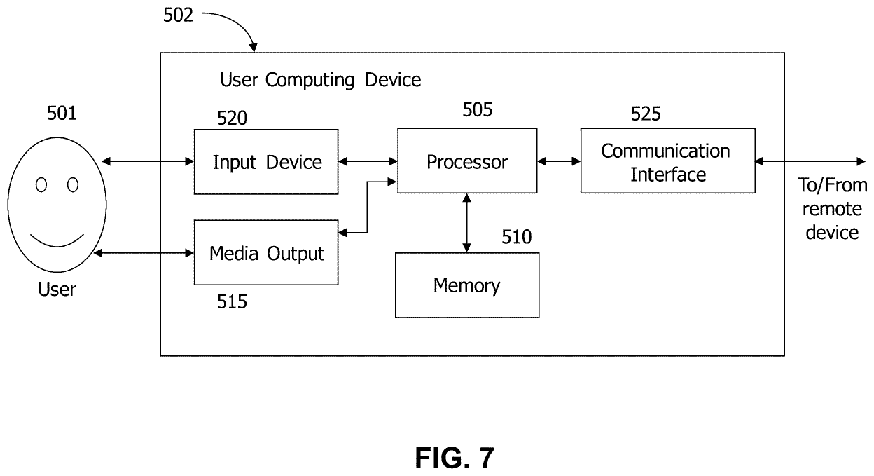

FIG. 7 depicts a configuration of a remote or user-computing device 502, such as the user computing device 330 shown in FIG. 5. Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G, or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

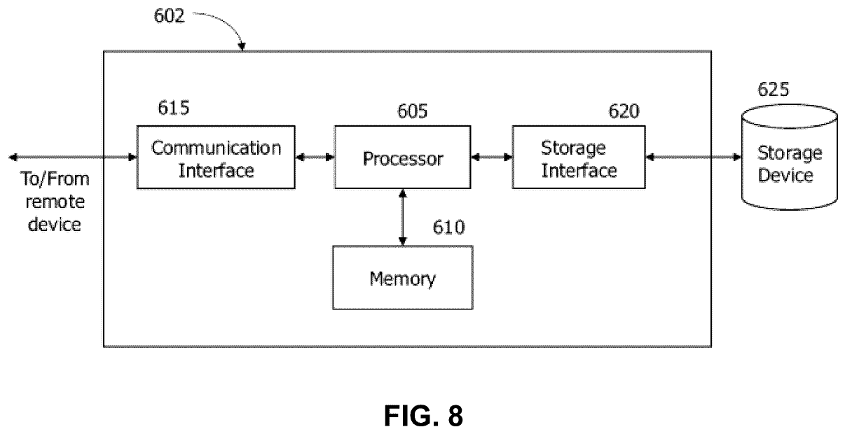

FIG. 8 illustrates an example configuration of a server system 602. Server system 602 includes, but is not limited to, database server 306 and computing device 302 (both shown in FIG. 5). In some aspects, server system 602 is similar to server system 304 (shown in FIG. 5). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 5) or another server system 602. For example, communication interface 615 may receive requests from user computing device 330 via network 350 (shown in FIG. 5).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 7) and 610 include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only, and are thus not limiting as to the types of memory usable for storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs further include: sequencing data, sensor data, image data, video data, telematics data, authentication data, authorization data, security data, mobile device data, geolocation information, transaction data, personal identification data, financial data, usage data, weather pattern data, “big data” sets, and/or user preference data. In some aspects, data inputs may include certain ML outputs.

In some aspects, at least one of a plurality of ML methods and algorithms may be applied, which include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically, ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

As will be appreciated based upon the foregoing specification, the above-described aspects of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving media, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor and can be implemented in a high-level procedural and/or object-oriented programming language, and/or in assembly/machine language. As used herein, the terms “machine-readable medium” and “computer-readable medium” refer to any computer program product, apparatus, and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application-specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are examples only, and are thus not limiting as to the types of memory usable for storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Washington). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independently and separately from other components and processes described herein. Each component and process can also be used in combination with other assembly packages and processes. The present aspects may enhance the functionality and functioning of computers and/or computer systems.

The methods and algorithms of the invention may be enclosed in a controller or processor. Furthermore, methods and algorithms of the present invention can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: WHOLE MAMMOGRAM IMAGE-BASED COX REGRESSION

To demonstrate the efficacy of a breast cancer risk prediction model that included analysis of mammogram images that were aligned and registered using the systems and methods disclosed hererin, the following experiments were conducted. A regression-based method (FLIP) was used to characterize a set of mammogram images from women undergoing routine screening and the characterized mammogram images were subjected to a standard survival analysis for risk prediction. Largely discarded data from standard digital mammograms were used to predict the 5-year risk of breast cancer using a Cox regression model.

Methods

Description of cohort. The Joanne Knight Breast Health Cohort (JKBHC) comprising over 10,000 women undergoing repeated mammography screening at Siteman Cancer Center and followed since 2010 was sampled to provide mammograms and additional data as described below for use in the experiments described below. All women obtained baseline mammograms at entry and completed risk factor questionnaires. Mammograms were all obtained using the same technology (Hologic). Women were excluded from the cohort if they had a history of cancer at baseline (other than nonmelanoma skin cancer). Women with breast implants were also excluded from the cohort. Follow-up through October 2020 was maintained through record linkages to electronic health records and pathology registries. 80% of participants had medical center visits (mammographies and other health visits) within the past 2 years.

All analyses performed in these experiments used the nested case-control cohort within JKBHC in which the pathology-confirmed breast cancer cases were matched to controls sampled from the prospective cohort based on the month of mammogram and age at entry. Women who were diagnosed within the first 6 months of baseline mammogram date were excluded in all analyses performed in the study (244 cases and 512 controls). Only craniocaudal (CC) views were used in this study, based on previous studies demonstrating superior 5-year risk prediction performance.

Image Processing. The CC-views (left and right) obtained from each woman were rotated to align the views in the same orientation. To minimize the noise caused by the distinct positions and sizes of individual breast regions, the mammograms were aligned using an automated bicubic interpolation algorithm as described above. In brief, the breast area within a raw mammogram was first segmented using a tight rectangular box, followed by soft tissue removal for parts outside of the breast. Each mammogram was then resized to 500×800 pixels using bicubic interpolation. After completion of the alignment as described above, the corresponding pixels for the aligned mammograms were averaged between the left and right sides at the baseline for this study. All images were de-meaned (centered) before the analytical procedures outlined below.

Statistical analysis. To develop an algorithm that directly accommodated mammogram images in a traditional Cox proportional hazards model, the aligned and registered mammogram images were characterized using a regression-based method that preserved the spatial distribution of informative features within the mammograms as described below.

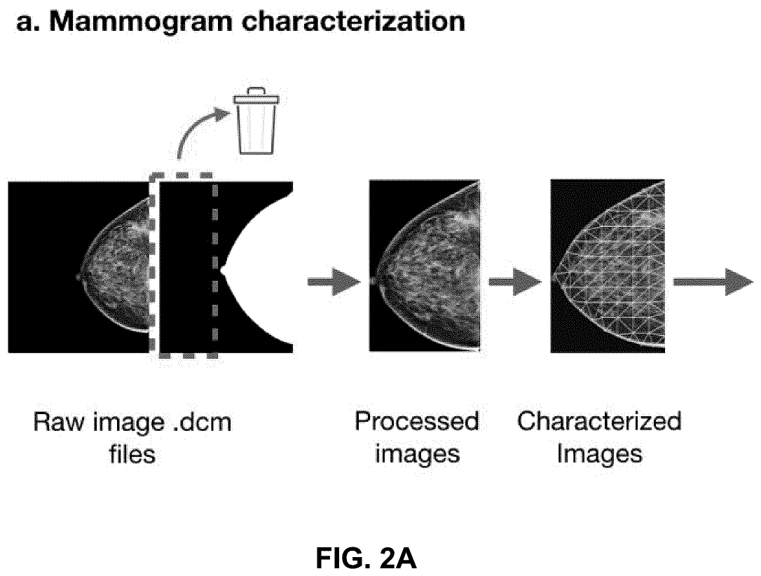

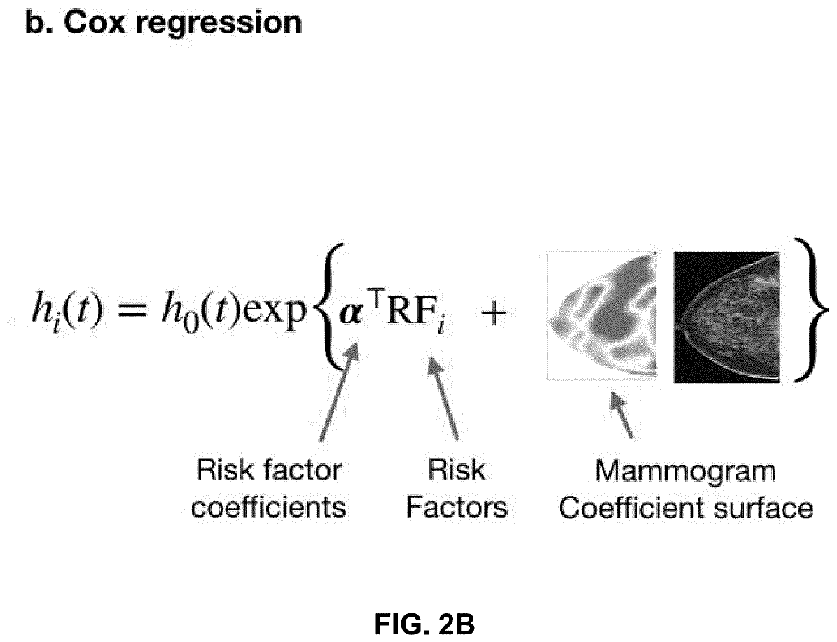

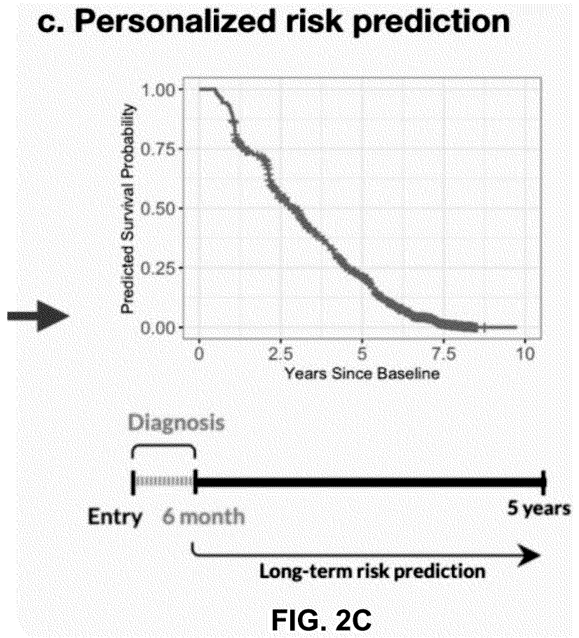

The regression-based framework (FLIP, functional model with image as predictor) was used to model the set of registered mammogram images from the patients. In brief, FLIP included three steps, illustrated in FIG. 2A, FIG. 2B, and FIG. 2C, respectively. As illustrated in FIG. 2A, FLIP received left and right craniocaudal (CC) mammogram views and averaged the pixels between the two sides after the images were aligned and registered using the systems and methods described herein. The mammograms analyzed using FLIP were treated as 2-dimensional (2D) objects instead of as long vectors of pixels to preserve the original spatial distribution associated with the original (raw) mammograms.

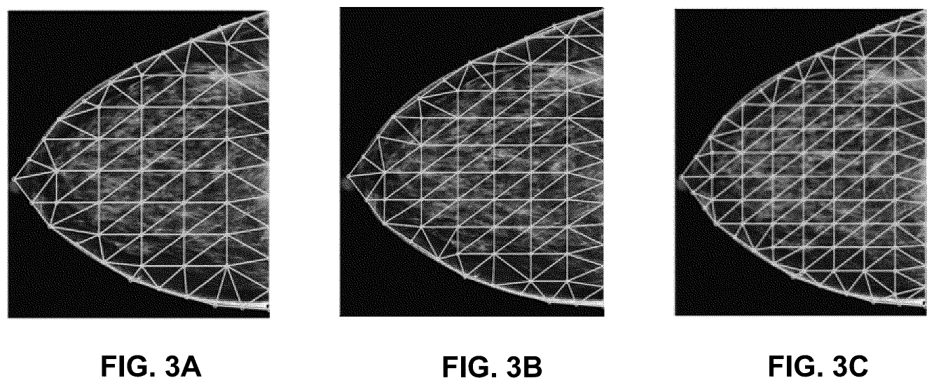

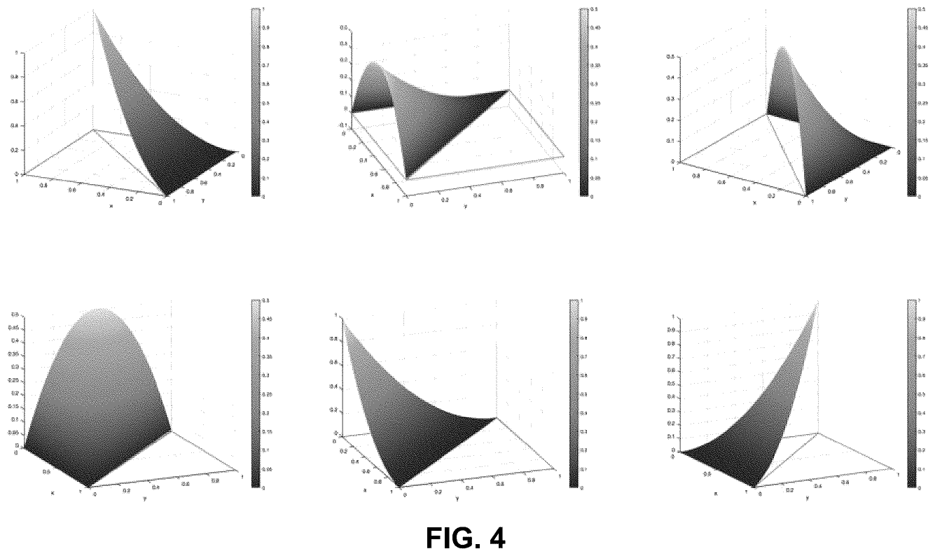

Referring again to FIG. 2A, the inputted and aligned/registered 2D mammograms were characterized with bivariate splines over triangulation to accommodate the irregular semi-circular breast regions. The breast areas within mammogram images were bounded in semi-circular regions. Bivariate splines that were piecewise polynomial functions defined over two-dimensional triangulated domains were used to approximate the mammograms, as illustrated in FIGS. 3A, 3B, and 3C by way of non-limiting examples.

Bivariate splines were obtained over a triangulation defined as Ω=Uⱼ₌₁^{j}τⱼ, comprising a collection of triangles Δ={τ₁, . . . , τⱼ} if any nonempty intersection between a pair of triangles in Δ was either a common vertex or a common edge; τ denotes a triangle that was a convex hull of three points that were not collinear. Degree d and smoothness r spline spaces were defined over the triangulation Δ: [symbol] _{d}^{r}(Δ)={z∈ [symbol] ^{r}(Ω):z|_{τ}∈ [symbol] _{d}, τ∈Δ}, where [symbol] ^{r}(Ω) was the collection of all rth continuously differentiable functions over Ω, for r≥0. The space of all polynomials with degree ≤d was denoted as [symbol] _{d} and thus z|_{τ} was the polynomial restricted on triangle τ. In some embodiments, a proper triangulation typically referred to triangulations containing well-shaped triangles with no small angles and/or obtuse angles. The triangulation grid was constructed using the Delaunay Triangulation using the Matlab function DistMesh.

Sensitivity analysis was carried out in selecting the number of triangles that were optimal for characterizing the mammogram images. By way of non-limiting example, FIGS. 3A, 3B, and 3C illustrate triangulations obtained using 87, 115, and 147 triangles, respectively.

The Bernstein polynomial basis function was used as the bivariate spline for the characterization of mammograms (see FIG. 4). For an arbitrary point s∈Ω, g₂, and g₃ were defined as the barycentric coordinates of the point s relative to the triangle τ. The barycentric coordinates of the point s were interpreted as masses placed at the vertices of the triangle τ. The masses were all positive if and only if the point was inside the triangle. The Bernstein basis polynomial of degree d for a point s relative to a triangle τ was then as
Bᵢⱼₖ^{τ,d}(s)=(i! j! k!)⁻¹ d! g₁ⁱg₂^{j}g₃^{k}, for i+j+k=d, where B(s)=(B₁(s), . . . , B_{M}(s))^{T} was a vector of degree d bivariate Bernstein basis polynomials for [symbol] _{d}^{r}(Δ), and M was the number of Bernstein basis polynomials.

In the literature it was generally believed that when the subject-level images were less smooth, considering lower order splines with r=1 and d=2 or 3, was sufficient. By way of non-limiting example, a Bernstein polynomial basis function of r=1 and d=2 is shown in FIG. 4. For these experiments, a Bernstein polynomial basis function of r=1, d=3 defined over 115 triangles was used (see FIG. 3B); these parameters proved to be sufficient for characterizing the mammogram images. The characterization was further optimized by ranking the spatial image characteristics by their association with the survival time. The solution for the characterization step was closed-form and unique.

A Cox regression was constructed that incorporated the whole mammogram images characterized as described above. Each whole mammogram image was denoted as Z, and s was used to denote the location of a particular pixel within each 2-dimensional (2D) image. In accordance with the triangulation notation, Ω denoted the 2D semi-circular domain within the mammograms.

n denoted individuals within the cohort. For each individual i, the pair (Tᵢ, δᵢ) denoted the observed survival outcome, where Tᵢ was the minimum of failure and censoring time Cᵢ, and δᵢ was the censoring indicator where δᵢ=1 indicated that the observed time Tᵢ was the failure time. In some embodiments, a Cox proportional hazards model was used for the right-censored survival data. A hazard function for individual i at some time t was built, as expressed by:

hᵢ(t)=h₀(t)exp(α^{T}RFᵢ+β₁ξᵢ₁+β₂ξᵢ₂+ . . . ), (1)

where h₀(t) was the nonparametric baseline hazard function, RFᵢ denoted the baseline risk factors including age, breast density (BI-RADS), BMI, menopausal status, number of children, family history, and history of pathology-confirmed benign breast disease. The vector a denoted the coefficients for these risk factors. The kth latent component Lk denoted the projection of the ith mammogram image Zᵢ(s) onto a latent space defined by the weight function ϕₖ(s), as expressed by:

ξᵢₖ=∫_{s∈Ω}Zᵢ(s)ϕₖ(s)ds, (2)

where k=1, . . . , ∞. The kth weight function ϕₖ(s) was estimated as a linear combination of Bernstein basis polynomials, as expressed by:

ϕₖ(s)=Σₘ₌₁^{M}wₖₘBₘ(s), (3)

where Bₘ(s) denoted the m^{th} Bernstein basis polynomial that approximated the image over m triangulations and wₖₘ was the weight function. The number of basis functions M was fixed as a function of the number of triangles and the degree of polynomial splines that did not require tuning.

By substituting (3) into (2), the kth latent component was written as:

ξᵢₖ=Σₘ₌₁^{M}wₖₘ∫_{s∈Ω}Zᵢ(s)Bₘ(s)ds, (4)

Eqn. (4) was used to estimate the set of weight functions wₖₘ. In some embodiments, once ξᵢ₁, ξᵢ₂, . . . , were estimated, the model as expressed in Eqn. (1) was used for estimating the hazard function by the standard partial likelihood approach under the Cox proportional hazards model.

The method as described above extended the functional partial least squares framework to accommodate the right-censored outcomes. The mean imputation method was adopted to overcome the right-censoring issue under the functional partial least squares framework. In some embodiments, if an event was observed for an individual (δ=1), \tilde{Y}ᵢ was set to f(Tᵢ). The function ƒ(⋅) was a transformation function that ensured that the observed time was on the real line. In some embodiments, the log transformation function was used. The unobserved failure times δᵢ=0 were replaced by their expected values, given that the failure time was larger than the censored time Cᵢ, as expressed by:

$${\tilde{Y}}_{i} = \frac{{Σ}_{R ( b ) > {C}_{t}} f ( {R}_{b} ) Δ S ( {R}_{( b )} )}{S ( {C}_{i} )} , \qquad ( 5 )$$

where R₍₁₎<R₍₂₎< . . . <R_{(B)} denoted the B ordered distinct failure times, S(⋅) was the Kaplan-Meier survival function of T, and ΔS(R_{(b)}) denoted the jump size of S(⋅) at time R_{(b)}. In this setup, the largest observation was treated as the true failure, amounting to making R_{(b)} the largest mass point of the estimated survival function of T.

The computation algorithm provided unique and closed-form solutions for the latent components ξᵢ₁, ξᵢ₂, . . . , for use in the Cox model. Taking the first set of basis coefficients w₁=(w₁₁, w_{1M})^{T} as an example,

cov²(ξ₁,\tilde{Y})=ξ₁^{T}\tilde{Y}\tilde{Y}∫₁, (6)

was maximized with the constraint that w₁^{T}w₁=1, where ξ₁=(ξ₁₁, ξₙ₁)^{T}, and \tilde{Y}=(\tilde{Y}₁, . . . , \tilde{Y}ₙ)^{T}. The solution to Eqn. (6) was unique and equal to w₁=(ZB)^{T}\tilde{Y}. The subsequent wₖ,k=2, . . . , was also chosen to maximize the covariates function subject to the constraint that wₖ^{T}wₖ=1 and wₖ^{T}wⱼ=0 if k≠j. A roughness penalty was added to satisfy the smoothness constraints under the functional setting. A unique and closed-form solution w₁=(I+λP)⁻¹(ZB)^{T}\tilde{Y} was obtained with P denoting a symmetric positive semi-definite penalty matrix and A denoting the smoothing parameter that can be chosen via cross-validation.

The model described above was used to generate a survival curve for individual patients, shown illustrated in FIG. 2C. Given the set of latent components estimated as described above, α for the baseline risk factors as well as β of length K as outlined in equation (1) were estimated. Specifically, Eqn. (1) was rewritten as:

where the coefficient surface for the mammogram image was denoted with c(s)=Σₖ₌₁^{K}βₖ ϕₖ(s),s∈Ω With this setup, the survival distribution at time t was written as:

S₀(t)^{exp}(α^{T}RFᵢ+β^{T}ξᵢ), (8)

under the proportional hazards assumption, where S₀(t)=exp(−∫₀^{t}h₀(u)du). The proportional hazards assumption was deemed reasonable upon formally inspecting the Schoenfeld residual plots for each of the baseline covariates.

It took about 6.28 minutes (377.03 seconds) to fit FLIP on the case-control cohort on a standard desktop without parallel computing (3.6 GHz Intel Core i9, 64 GB RAM). Given the fitted FLIP, it took less than about 5 seconds to output an individualized projected future risk. The computational time reported above did not include image processing time. The computational speed may be further optimized using parallel computing methods.

The use of the FLIP analysis method was accompanied by at least several beneficial properties, including simplicity, robustness, transparency, and ease of interpretation of hazards/hazard ratios. The transparent workflow included a Cox model that ensured high reproducibility across other studies. FLIP generated unique and closed-form solutions. FLIP did not rely on prohibitive training data or extensive computational requirements. FLIP offered a standard statistical solution to the big data challenge posed by mammogram images. The analyzed images, whole mammograms, reflected universal biologic mechanisms. Prospectively collected data were used to evaluate performance. The image analysis methods described above enabled information extraction from complex multidimensional data for managing, interpreting, and visualizing the 2D mammograms and 3D tomosynthesis images. In some embodiments, the image analysis methods described above provided instantaneous solutions for medical image registration and alignment.

The characterization was further optimized under the computation algorithm described above (see Eqn. (6)) such that the spatial image characteristics were ranked by their association with the survival time. The solution within this step was not only closed-form but also unique which ensured reproducibility across different studies. As shown in FIG. 2B, a standard Cox regression was fit using the whole mammogram image as an additional risk factor in addition to existing factors such as age, breast density (BI-RADS), BMI, menopausal status, number of children, family history of breast cancer, and history of pathology confirmed benign breast disease. The proportional hazards assumption was deemed reasonable upon formally inspecting the Schoenfeld residual plot for each of the baseline covariates.

All models were evaluated using Uno's estimator of cumulative 5-year AUC for right-censored time-to-event data. To assess the prediction performance, a 10-fold internal cross-validation was performed using the 756 women by randomly partitioning the case-control cohort into 10 subsamples. The dataset under each cross-validation was fixed to be the same for all models to ensure a consistent basis of comparison. Within the training sample under each fold, ⅓ of the women were randomly selected as the development dataset for selecting the tuning parameters. The optimal tuning parameters (smoothness penalty of the bivariate splines for triangulation and the number of latent components used to characterize the images) were determined via an automated two-dimensional grid search such that the 5-year AUC was optimized for a given set of tuning parameters in the development dataset.

To assess the significance of the difference between the two AUCs (baseline vs. disclosed model), the likelihood ratio test was used between the two nested models for assessing the incremental predictive information with the addition of mammogram images.

Results

Overview of the proposed method. The Cox proportional hazards model is one of the most widely used methods for survival analysis. Many well-developed breast cancer risk prediction models build on the Cox regression for its simplicity, robustness, transparency, and ease of interpretation of hazards/hazard ratios. Intuitively, one can adopt the Cox model to facilitate image-based risk prediction by making full use of the mammograms at the baseline. However, a regression-based model involving millions of pixels (˜13 million pixels per digital mammogram) in general was impractical, as the total number of model coefficients would greatly exceed the number of women. To effectively characterize the mammograms for a standard survival analysis for risk prediction using Cox regression, the FLIP model (functional model with image as predictor), described above, was used

The proportional hazards assumption was formally checked by inspecting the Schoenfeld residuals for all baseline covariates. With the Cox regression, the personalized long-term risk was easily forecasted as the final step of FLIP in less than 5 seconds.

Evaluating prediction performance within the Joanne Knight Breast Health Cohort 124 (JKBHC), FLIP was fitted and cross-validated in the case-control cohort within the JKBHC of women without a history of breast and other cancers at recruitment during routine mammography screening from 2008 through 2012 with mean age 57 years, 73% postmenopausal, 79% White, 5.7% BI-RADS D (dense breast) 4th edition. The median time of follow-up was 6.27 (SE 2.32) years and the median time to diagnosis since baseline was 5.19 (SE 2.42) years.

To assess the prediction performance of the proposed algorithm, a 10-fold cross-validation was performed which involved randomly partitioning the case-control cohort into 10 subsamples. A base model was first constructed with data that were routinely available at screening mammography that included age and density (BI-RADS), and then the whole mammogram image (WMI) was added to assess the improvement in prediction. The 5-year AUC averaged between the cross-validation from the base model increased from 0.55 to 0.68 with WMI added. Then BMI and menopausal status were added which are also routinely available from women at screening mammography. In this model with routine clinic data, the 5-year AUC for the base model increased from 0.64 to with WMI added. Finally, to reflect the potentially richer data on questionnaire risk factors that might further improve the base model, history of childbirth (yes/no), history of benign breast disease confirmed by biopsy (yes/no), and family history of breast cancer (yes/no) were added. We note that the prediction performance did not improve with these added risk factors over the simpler model, and the addition of WMI again increased the AUC from 0.63 to 0.70. All three models with the added WMI were significantly improved (P<0.001) from the base models.

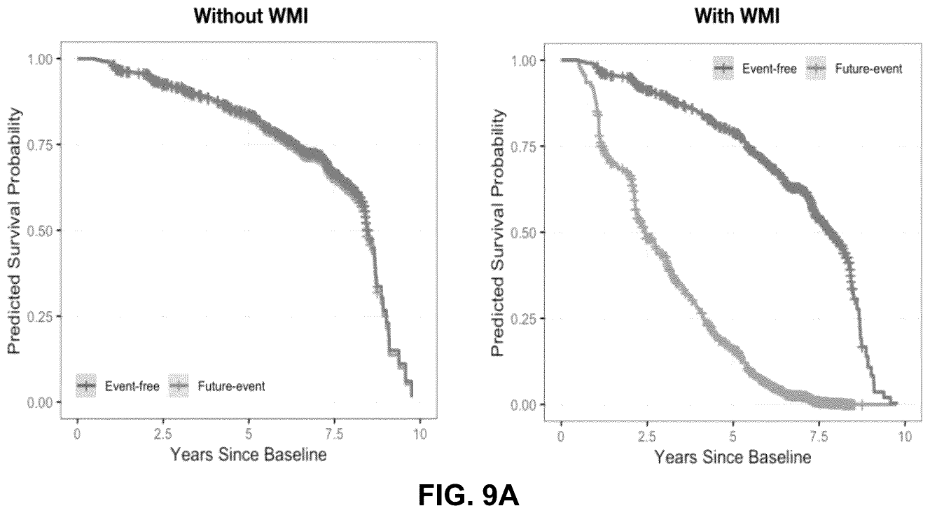

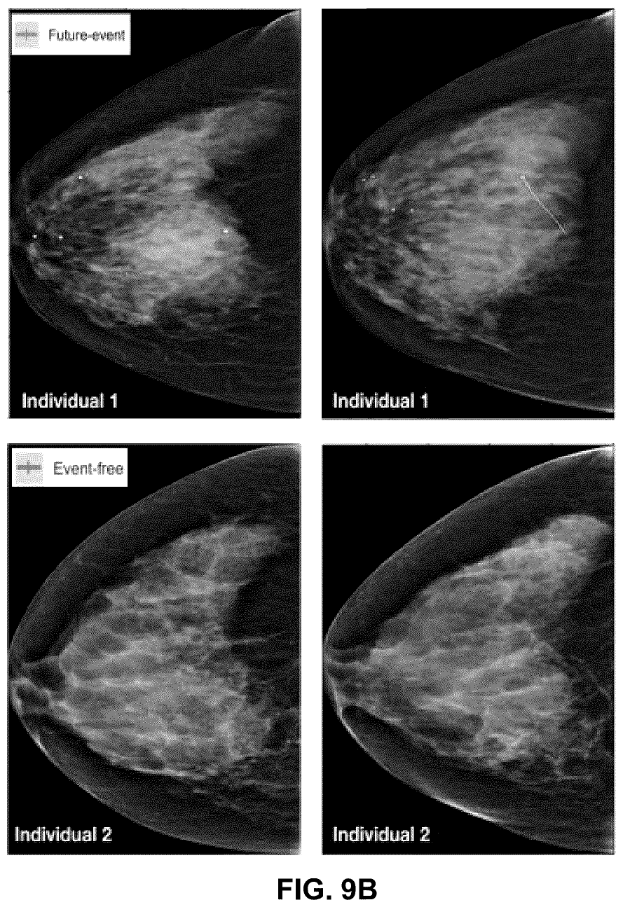

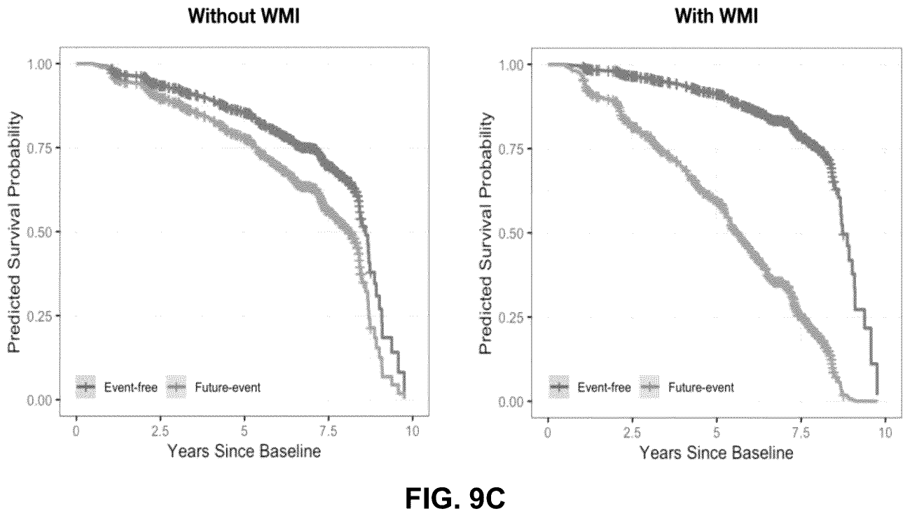

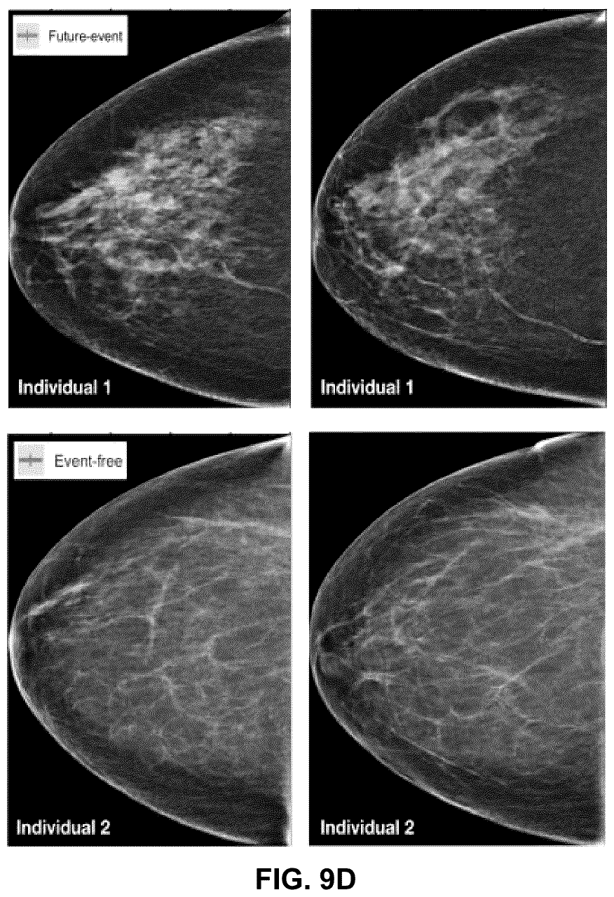

Forecasting personalized survival probability. To demonstrate the value of adding the WMI to the prediction model, the projected personalized survival probability is plotted in FIG. 9A for 2 randomly selected women in the testing dataset with extremely dense breasts (BI-RADS category D; highest risk). These women aged 50-59.9, postmenopausal, had a history of benign breast biopsy, and family history and parity as noted in FIG. 9. Without WMI in the Cox regression, the predicted survival probability free from breast cancer is inseparable for these two women. However, a marked separation in the predicted survival curves is observed after adding in the WMI (right panel), reflecting the improved AUC for FLIP. Comparable survival curves are presented in FIG. 9C for two randomly selected women in the testing dataset with breasts with scattered fibroglandular density (BI-RADS category B) and again, a marked separation with the addition of the WMI in the Cox regression is observed. Here, in addition to the higher predicted risk for the woman with a future event, we see that the event-free woman is shown to have a lower predicted risk over time when WMI was added into the Cox regression. This is critical in the prevention context to identify low-risk women who may need less intensive screening or surveillance and can be guided to risk-appropriate programs.

Secondary analysis. The AUCs for different prediction time horizons from 2 to 5 years are presented in Table 1 below:

TABLE 1
Breast Cancer Risk Prediction Comparison

| | | Base model | | | | Base model + WMI | | | |
|---|---|---|---|---|---|---|---|---|---|
| | Co- | Year | Year | Year | Year | Year | Year | Year | Year |
| Model | variates | 2 | 3 | 4 | 5 | 2 | 3 | 4 | 5 |
| Baseline | Age + density | 0.50 | 0.54 | 0.55 | 0.55 | 0.70 | 0.68 | 0.68 | 0.68 |
| | (BI-RADS) | (0.06) | (0.03) | (0.03) | (0.02) | (0.05) | (0.04) | (0.04) | (0.03) |
| Clinical Data | Age + | 0.69 | 0.67 | 0.65 | 0.64 | 0.75 | 0.73 | 0.73 | 0.72 |
| | density | (0.07) | (0.05) | (0.05) | (0.04) | (0.05) | (0.04) | (0.04) | (0.04) |
| | + | | | | | | | | |
| | menopause + | | | | | | | | |
| | BMI | | | | | | | | |
| Clinical + | Clinical | 0.68 | 0.66 | 0.65 | 0.63 | 0.74 | 0.70 | 0.70 | 0.70 |
| reproductive | data + | (0.06) | (0.05) | (0.05) | (0.04) | (0.05) | (0.04) | (0.04) | (0.04) |
| data | parity | | | | | | | | |
| | (yes/no) + family | | | | | | | | |
| | history + BBD | | | | | | | | |

As expected, a general trend of increase in the mean AUC averaged over the 10-fold cross-validation is observed, and a bigger standard error with a shorter prediction horizon. In the model with age, BI-RADS, and clinical data, for example, the AUC increased from 0.72 (SE 0.04) for the 5-year prediction to 0.75 (SE 0.05) for the 2-year prediction. To confirm the model performance across risk factors and breast cancer subtypes, analysis limited to invasive breast cancer was repeated, to postmenopausal women vs premenopausal, and white women vs black. The AUC showed no meaningful difference in these subgroups from the overall results presented above. For postmenopausal women (553 women with 176 cases), the AUC for the base model increased from 0.64 to 0.69 with WMI added. For invasive breast cancer (169 cases), the model with all risk factors increased from 0.66 to 0.69 when the WMI is added. For white women (190 cases), the AUC for the base model was 0.63 and increased to 0.68 with WMI. For black women (49 cases), the AUC was 0.63 in the base model and increased to 0.69 with WMI added to the prediction model. All comparisons between the baseline and the proposed model across risk factors and breast cancer subtypes are statistically significant (P<0.001).

### Example 2: Pectoral Muscle Removal in Mammogram Images: A Novel Approach for Improved Accuracy and Efficiency

### Abstract

Purpose: To evaluate the performance of the approach described herein to remove pectoral muscles from mediolateral oblique (MLO) view mammograms, the following experiments were conducted.

Methods: A pectoral muscle identification pipeline was developed, first image was binarized to enhance contrast, then the Canny algorithm was applied for edge detection. The accuracy of pectoral muscle identification was assessed using 951 women (1902 MLO mammograms) from the Joanne Knight Breast Health Cohort at Washington University School of Medicine. “False positives” (FP) are defined as regions that are incorrectly identified as pectoral muscle despite being outside of the true region, and “false negatives” (FN) as regions within the true region that are erroneously identified as breast tissue. Performance is compared to Libra.

Results: On average, the disclosed algorithm exhibited a lower mean error of 8.22% in comparison to Libra's estimated error of 14.44%. Evaluating by type of error (false positive (FP) and false negative (FN)), it is shown that Libra tends to overestimate the FP by 25.83% compared to the disclosed algorithm of 4.17%. On the other hand, the disclosed algorithm tends to overestimate the FN by 12.23% compared to Libra of 3.04%.

Conclusions: A novel approach for pectoral muscle removal in mammogram images is presented that demonstrates improved accuracy and efficiency compared to existing methods. The findings have important implications for the development of computer-aided systems and other automated tools in this field.

### Introduction

Breast cancer is a leading cancer among women worldwide, accounting for 1 in 4 cancers diagnosed in women. The social and economic impact of this cancer underscores the importance of early detection and effective treatment. Mammography, a widely used for breast cancer screening, and typically involves acquiring two different views—the craniocaudal (CC) view and the mediolateral oblique (MLO) view. The CC view is obtained by imaging the breast from a superior to inferior direction, while the MLO view is acquired from a lateral oblique angle which includes parts of the pectoral muscle from the chest that overlaps with the breast tissue. As we move to the global use of digital mammography and increasingly need to integrate multiple exams over time to improve performance, efficient image processing and alignment are increasingly important.

Pectoral muscle removal, or segmentation, is a critical step in many computer-aided systems. In mammographic density estimation, for example, accurate removal of pectoral muscle is crucial in obtaining the correct dense tissue area/volume with respect to the total breast size. Automated diagnostic tools, on the other hand, also face challenges in the analysis of breast tissue due to the presence of the pectoral muscle. This is particularly evident in the upper outer quadrant of the breast where the pectoral muscle can introduce increased noise, potentially interfering with the accuracy of image analysis. Thus, in the development of intricate pipelines for automated or computer-aided algorithms of breast tissue evaluation or cancer detection, the removal of the pectoral muscle is often considered a vital initial step that requires careful attention and prioritization.

In a recent study, a comparison was made between two commonly used methods, namely Libra and OpenBreast for pectoral muscle removal in full-field digital mammogram (FFDM) images. That study included 168 women revealing that Libra exhibited superior performance in 4 terms of accuracy when compared to OpenBreast. Our work, on the other hand, presents a novel approach that further improves the current methodology in pectoral muscle removal.

Through extensive evaluation of a large dataset of 951 women with 1,902 MLO-view mammograms, we demonstrate a superior accuracy in identifying the pectoral muscle from FFDM mammogram images, along with improved overall efficiency in terms of computational time, when compared to Libra. Our findings offer a promising solution for enhanced image analysis in the context of breast tissue evaluation and mass detection, providing valuable insights for further advancements in the field.

### Method

### Study Population

The Joanne Knight Breast Health Cohort (JKBHC) consists of over 10,000 women who undergo repeated mammography screening at Siteman Cancer Center and have been followed since 2010. All women in the cohort had a baseline mammogram at entry and completed a risk factor questionnaire. Full-field digital mammograms were obtained using the same technology (Hologic). Women with a history of cancer at baseline (except nonmelanoma skin cancer) were excluded from the cohort. Follow-up data until October 2023 were obtained through record linkages to electronic health records and pathology registries, as previously described. Approximately 80% of participants had a medical center visit, including mammography and other health visits, within the past 2 years. All analyses performed in this study use the nested case-control cohort within JKBHC, where the pathology-confirmed breast cancer cases were matched to two controls sampled from the cohort based on a month of mammogram and age at entry. After excluding women with breast implants, and those with missing mammography images, 294 cases and 657 controls were retained. As the pectoral muscle only appears in the 5 mediolateral oblique (MLO) view full-field digital mammograms on the left and right breasts, a total of 1,902 images were analyzed.

### Pectoral Muscle Identification Algorithm

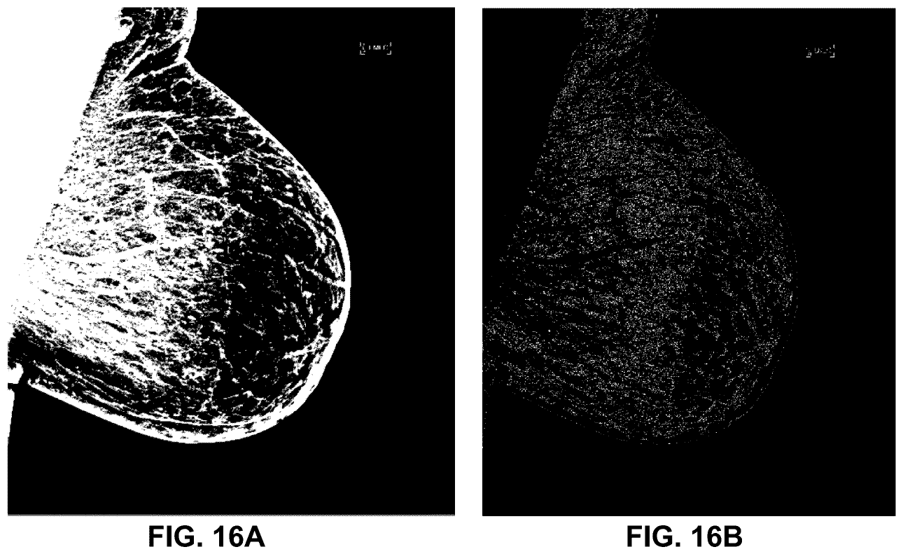

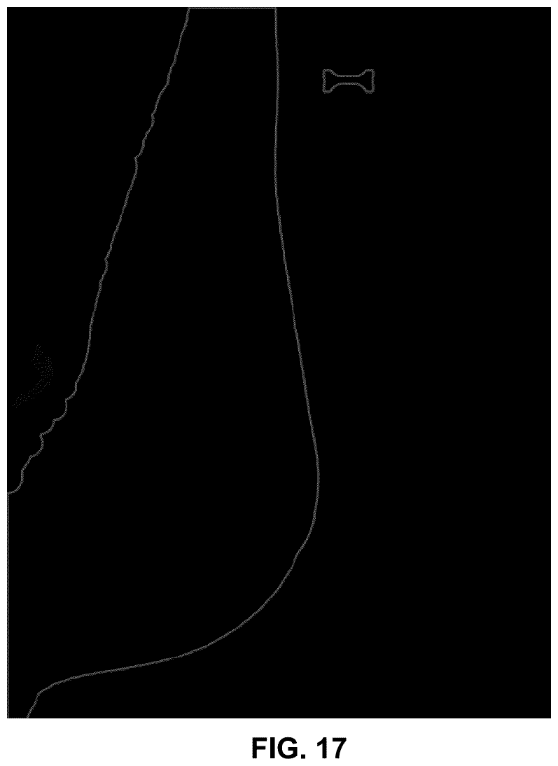

The proposed pectoral muscle identification pipeline is as follows. Initially, the image is subjected to binarization to enhance contrast. This process amplifies the distinction between highly bright pixels in the breast to less prominent ones; see FIG. 16A as an example. Following binarization, we applied the Canny algorithm for edge detection where a rough outer edge of the breast, excluding the pectoral muscle region, was found, as illustrated in FIG. 16B. Note that the detected edge of the breast is on the pixel level and does not yet present a smooth edge. It is thus proposed to adopt a robust interpolation to smooth all the discontinuous regions presented within the mammogram. As depicted in FIG. 17, the periphery of the breast tissue is well estimated with the proposed algorithm. Because the algorithm automatically detects the breast tissue, the pectoral muscle, as a result, is consequently identified.

### Statistical Approach

“False positives” (FP) are defined as regions that are incorrectly identified as pectoral muscle despite being outside of the true region, and “false negatives” (FN) as regions within the true region that are erroneously identified as breast tissue. The percentage of total pixels that make up the false positives (FP) and false negatives (FN) with respect to the true pectoral muscle regions on each mammogram is estimated. False positive (FP) and false negative (FN) findings are summarized for both the proposed method and for the application of Libra to the study images.

### Results

The accuracy of pectoral muscle identification was estimated using 951 women containing both the left and right MLO-views, resulting in a total of 1,902 mammograms. The risk factor profile for these women has been reported previously. Women are Black (15%) white (81%) or other race/ethnicity. The mean age is 57 and 73% are postmenopausal.

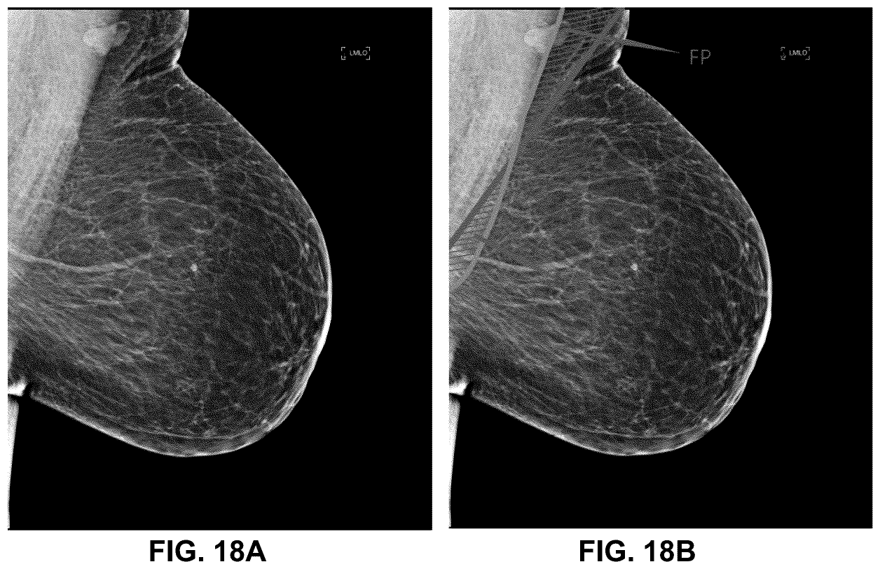

Two distinct types of errors that can occur during the pectoral muscle identification progress were first demonstrated, as illustrated in FIG. 18. Specifically, with reference to the true pectoral muscle region, indicated by the green line in FIG. 18, “false positives” (FP) were defined as regions that were incorrectly identified as pectoral muscle despite being outside of the true region, and “false negatives” (FN) were defined as regions within the true region that were erroneously identified as breast tissue.

The percentage of total pixels that make up the false positives (FP) and false negatives (FN) with respect to the true pectoral muscle regions on each mammogram are estimated. Because prior findings identified Libra to be superior in terms of accuracy when compared to OpenBreast, the disclosed algorithm was compared with Libra in this section. Both the FP and FN errors were investigated using both the proposed method and Libra on the same set of 1,902 images.

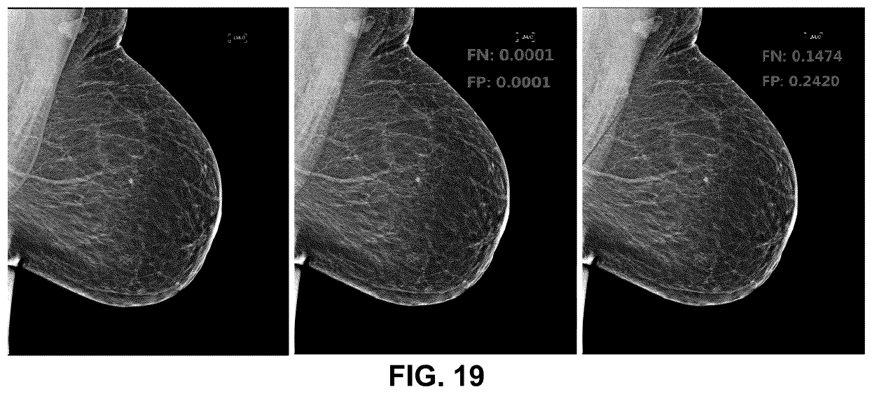

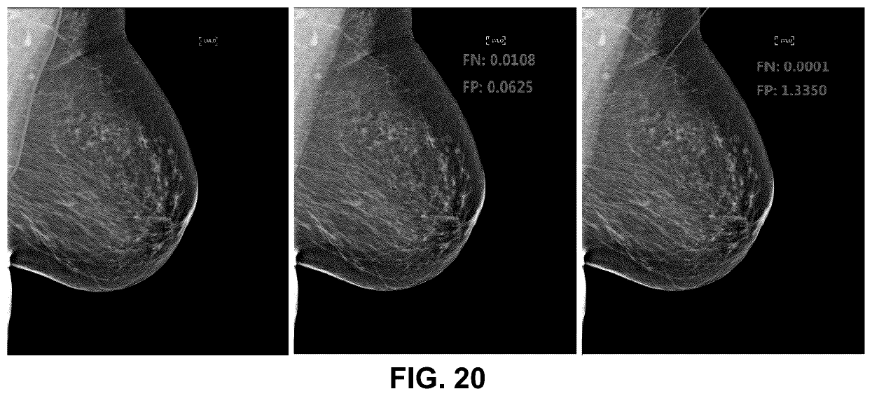

For visualization purposes, two examples are first shown in FIG. 19 and where the first column represents the true pectoral muscle regions. The identified pectoral muscle region is shown using the disclosed algorithm (second column) in comparison to Libra (last column) with their corresponding false positive and false negative errors reported on each. In both examples, it can be seen that the pectoral muscle identified using the proposed algorithm is very close to the true region where the errors are hardly noticeable by the naked eye. Libra, on the other hand, tends to overestimate the pectoral muscle region by including areas that are within the breast.

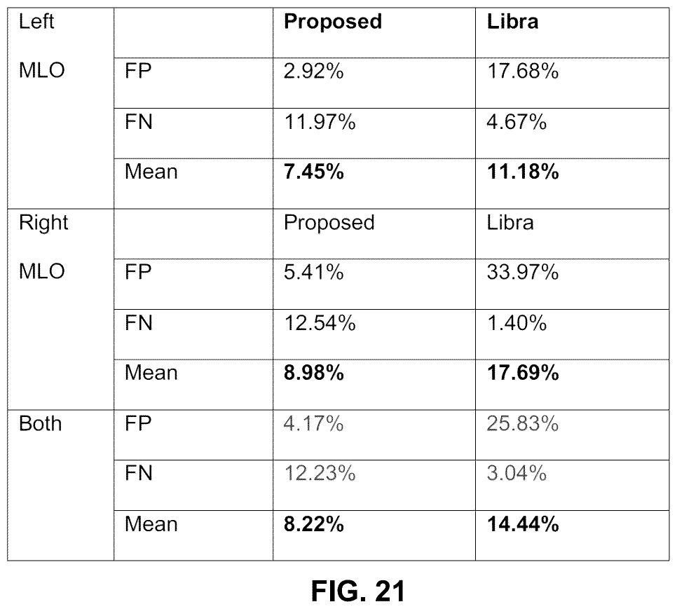

The results from applying the proposed method and Libra over all 1,902 MLO mammograms are shown in FIG. 21. It is seen that on average, the disclosed algorithm exhibits a lower mean error of 8.22% in comparison to Libra's estimated error of 14.44%. That is, the disclosed algorithm minimizes 43% of the error compared to Libra when looking at the true positive and true negative regions together.

When separated by type of error (FP and FN), Libra typically overestimated the FP by 25.83% compared to the disclosed algorithm estimate of 4.17%. On the other hand, the disclosed algorithm overestimated the FN by 12.23% compared to the Libra overestimate of 3.04%.

Furthermore, the algorithm demonstrated significantly improved processing speed compared to Libra. When tested on the same dataset, the algorithm takes, on average, 2 seconds to output the pectoral muscle region, whereas Libra takes approximately 20 seconds. This suggests an approximately times efficiency gain in computational time, which could significantly speed up future needs in pectoral muscle identification in other computer-aided algorithms.

### Discussion

The study draws on routine screening mammograms from a prospective cohort and introduces a novel and efficient approach for pectoral muscle removal in full-field digital mammogram images that demonstrated improved accuracy and efficiency compared to Libra. The findings of the study have important implications for computer-aided systems and other automated tools used in breast cancer screening, diagnosis, and risk prediction. One of the key challenges in developing computer-aided systems in breast tissue evaluation and mass detection is the accurate removal of the pectoral muscle within MLO-view mammograms, which can interfere with the analysis of breast tissue. The extensive evaluation of a large dataset of 951 women with 1,902 MLO-view full-field digital mammogram images demonstrated the superior accuracy of the approach in identifying the pectoral muscle, thereby reducing the risk of false positive or false negative muscle removal in subsequent image analysis. Furthermore, the approach also offers enhanced efficiency in terms of computational time compared to existing methods. The reduced computational time is a significant advantage, as it can improve the overall performance of computer-aided systems by reducing processing time and increasing throughput, which is crucial for real-time or near-real-time applications in clinical settings.

Other studies have acknowledged the challenge of pectoral muscle removal. Studies of digitized screening film mammograms have manually removed pectoral muscle and noted that consistency among different readers is not a straightforward task. Others have used computer programs to remove muscle from CC but not from MLO views.

### CONCLUSION

The study presents a novel approach for pectoral muscle removal in mammogram images that demonstrates improved accuracy and efficiency compared to existing methods. The findings contribute to the growing body of literature on image analysis for breast cancer screening and diagnosis, and contribute to the development of computer-aided systems and other automated tools in this field.

What is claimed is:

1. A system for aligning and registering a medical image with a reference medical image, the system comprising at least one processor in communication with at least one memory device, wherein the at least one processor is programmed to:
a. receive the medical image and a reference image;
b. convert the medical image to a binary image;
c. isolate an area of interest within the medical image to produce an isolated image;
d. remove at least one portion of the isolated image containing at least one user-selected tissue type to produce a segmented image;
e. flip or rotate the segmented image into alignment with the reference image to produce an aligned image; and
f. register the aligned image to the reference image to produce an aligned and registered image.

2. The system of claim 1, wherein the medical image is selected from a longitudinal series of medical images and the reference image comprises an initial medical image of the series.

3. The system of claim 1, wherein the medical image is selected from a dataset comprising a plurality of medical images obtained from a plurality of subjects and the reference image comprises a user-selected medical image from the dataset.

4. The system of claim 1, wherein the medical image is selected from a digital mammogram image and at least a portion of a digital 3D tomosynthesis image.

5. The system of claim 1, wherein the medical image further comprises a craniocaudal view or a mediolateral oblique view.

6. The system of claim 5, wherein the area of interest of the medical image comprises a portion of the medical image containing a breast region.

7. The system of claim 6, wherein the area of interest is isolated by fitting a rectangle of minimal dimension around the breast region.

8. The system of claim 7, wherein the at least one user-selected tissue type removed from the isolated image comprises soft tissues outside of the breast region within craniocaudal views, pectoral muscle tissue within mediolateral oblique views, and any combination thereof.

9. The system of claim 8, wherein the at least one processor is further programmed to automatically determine the soft tissues outside the breast region based on a union of discontinuities on a boundary of the breast area and deviations from a semi-circular shape, wherein the semicircular shape is selected to approximate the boundary of the breast area.

10. The system of claim 8, wherein the at least one processor is further programmed to automatically determine the pectoral muscle tissue by binarizing the medical image, applying a Canny algorithm to detect an outer edge of the breast tissue, and removing a portion of the image falling outside of the outer edge of the breast tissue.

11. The system of claim 1, wherein the at least one processor is further programmed to produce the aligned image by:
a. finding a width ratio between the segmented image and the reference image;
b. obtaining an alignment angle between a line along the top of the segmented image and a line connecting the top left corner and the largest horizontal (x) point of the breast tissue within the segmented image; and
c. rotating the segmented image to align the alignment angle with a corresponding alignment angle of the reference image.

12. The system of claim 1, wherein the at least one processor is further programmed to register the aligned image to the reference image by adjusting a ratio in image width pixelwise between the aligned image and the reference image.

13. The system of claim 2, wherein the at least one processor is further programmed to:
a. identify an abnormal region within one medical image from the longitudinal series of medical images;
b. identify a monitor region for each medical image of the longitudinal series of medical images, wherein the monitor region of each medical image is matched to the abnormal region of the one medical image; and
c. display a series of monitor images to a user, the series of monitor images comprising the longitudinal series of medical images demarcated with each corresponding abnormal region or monitor region.

14. The system of claim 12, wherein the at least one processor is further programmed to display magnified views of the abnormal region and monitor regions to the user.

15. The system of claim 1, wherein the at least one processor is further programmed to:
a. identify text within the medical image; and
b. determine a view of the binary image based on the identified text, wherein the view is a craniocaudal view or a mediolateral oblique view.

16. A system for predicting a risk of breast cancer of a patient from analysis of a medical image, the system comprising at least one processor, the at least one processor configured to:
a. transform the medical image into a characterized image by forming bivariate splines over a two-dimensional triangulated domain of the medical image;
b. perform a survival analysis of the characterized image to obtain a prediction of the risk of breast cancer in the patient; and
c. display the prediction of the risk of breast cancer to a practitioner.

17. The system of claim 16, wherein the at least one processor is further configured to form bivariate splines over a two-dimensional triangulated domain of the medical image by forming the two-dimensional triangulated domain using Delaunay Triangulation and forming the bivariate splines using a Bernstein polynomial basis function.

18. The system of claim 16, wherein the at least one processor is further configured to perform a survival analysis of the characterized imaging using a model selected from a right-centered survival model and a Cox proportional hazards model.

19. The system in claim 16, wherein the medical image is a mammogram.
